(19) United States
(12) Patent Application Publication
(10) Pub. No.: US 2018/0304026 A1
(43) Pub. Date: Oct. 25, 2018

(54) DRUG DELIVERY DEVICE WITH DISPENSING FEEDBACK

(71) Applicant: SANOFI, Paris (FR)

(72) Inventors: Anthony Paul MORRIS, Coventry (GB); William MARSH, Buckingham (GB); Matthew Meredith JONES, Warwick (GB)

(21) Appl. No.: 15/758,603

(22) PCT Filed: Aug. 8, 2016
(86) PCT No.: PCT/EP2016/068858

§ 371 Date: Mar. 8, 2018

(30) Foreign Application Priority Data
Sep. 9, 2015 (EP) 15306371.4
Apr. 6, 2016 (EP) 16305395.2

(51) Int. Cl. A61M 5/315; A61M 5/20

(52) U.S. Cl. CPC A61M 5/31553; A61M 5/20; A61M 5/31583; A61M 2205/581; A61M 2205/582; A61M 5/3157

(57) ABSTRACT

The disclosure relates to a drug delivery device for mechanically dispensing a dose of a medicament. The drug delivery device includes a ratchet mechanism adapted to emit an audible and/or tactile feedback during dispensing the dose of medicament. The ratchet mechanism includes a flexible arm and a toothed profile. A free end of the flexible arm is configured to engage with the toothed profile and to perform a relative rotational movement thereto during dispensing the dose of medicament, thereby generating the audible and/or tactile feedback, and the flexible arm extends in a plane parallel to a rotational axis of the flexible arm.

### CROSS REFERENCE TO RELATED APPLICATIONS

The present application is the national stage entry of International Patent Application No. PCT/EP2016/068858, filed on Aug. 8, 2016, and claims priority to Application No. EP 15306371.4, filed on Sep. 9, 2015, and Application No. EP 16305395.2, filed on Apr. 6, 2016, the disclosures of which are expressly incorporated herein in entirety by reference thereto.

### TECHNICAL FIELD

The disclosure relates to a drug delivery device.

### BACKGROUND

In certain types of drug delivery devices, such as pen type devices, pre-filled cartridges are used. These cartridges are housed in a cartridge holder or cartridge housing. To dispense a certain set dose of a medicament contained in such a cartridge, the drug delivery device has a dose setting element.

During drug delivery, a piston rod coupled to the dose setting element presses against a plunger contained within the cartridge in order to dispense the medicament through an attached needle assembly.

A non-visual indication during drug delivery would be helpful in particular for visually impaired users of the drug delivery device. This may include an audible and/or tactile feedback during dose setting, dose correction, dose dispensing and/or at the end of dose dispensing.

Thus, there remains a need for an improved drug delivery device providing a useful feedback to a user during drug delivery.

### SUMMARY

In certain aspects, An improved drug delivery device providing a useful feedback to a user during drug delivery is provided.

A drug delivery device for mechanically dispensing a dose of a medicament comprises a ratchet mechanism adapted to emit an audible and/or tactile feedback during dispensing the dose of medicament, wherein the ratchet mechanism includes a flexible arm and a toothed profile, wherein an end portion of the flexible arm being configured to engage with the toothed profile and to perform a relative movement thereto while generating the audible and/or tactile feedback, and wherein the flexible arm extends in a plane perpendicular to the direction of the relative movement between the flexible arm and the toothed profile.

In an exemplary embodiment, a free end of the flexible arm is configured to engage with the toothed profile and to perform a relative rotational movement thereto while generating the audible and/or tactile feedback. The flexible arm extends in a plane running parallel to a rotational axis of the flexible arm.

Here, the rotational axis is an axis about which the flexible arm rotates. This rotational axis could be a rotational axis of a component of the drug delivery device on which the flexible arm is arranged. In other words: The flexible arm extends in an axial direction parallel to the axis of relative rotation between the flexible arm and the toothed profile. The flexibility of the flexible arm can be adjusted, i.e. in position, in particular in height or in an angle. The adjustment of the flexible arm enables a useful audible and/or tactile feedback for a user during dose dispensing and the possibility to change or correct a selected dose before dose dispensing in a non-destructive manner, due to the length from the fixed base of the flexible arm to a contact surface on the free end, and the angle of the contact surface providing a component of force to cause the flexible arm to deflect, thereby providing a high mechanical stability. Thus, the flexible arm extending axially parallel to the rotational axis enables translating torque in a rotational direction opposite to a rotational direction for dose setting in order to correct the set dose without damaging the flexible arm.

As mentioned above, the ratchet mechanism is configured as a rotational ratchet mechanism, wherein the flexible arm and the toothed profile are allowed to rotate with respect to each other during dispensing the dose of medicament. In an exemplary embodiment, the toothed profile may be configured as a gear chain and the flexible arm as a cantilever beam that engages the teeth of the gear chain. In particular, the toothed profile may be formed as a ring of inner or outer ratchet teeth, i.e. outer or inner circumferential spur gear toothing. Alternatively, the toothed profile may be formed as an axial toothing.

In an exemplary embodiment, the drug delivery device further comprises a cartridge adapted to contain the medicament and received within a housing, a depressable button operable to effect dose dispensing, a dose selector component operable to set or unset a dose of medicament before dose dispensing, a drive component adapted to push the medicament out of the cartridge, and a clutch element arranged between the dose selector component and the drive component. The dose selecting component may comprise a dial grip and a dose indicator sleeve that is marked with a sequence of numbers, which are visible through a window arranged within the housing to indicate the set dose. During dose setting, the dial grip is rotated and thus the dose indicator sleeve and the clutch element are rotated with respect to the drive component and the button. After setting the dose, the button can be pressed by a user causing an axial translation of the button and the clutch plate with respect to the dose selecting component. During dose dispense, the dose indicator sleeve, the clutch element and the drive component rotate with respect to the button. Thus, the clutch plate is configured as a connective link between the drive component and the dose selector component and may comprise a number of spline features to enable or prevent relative movement between the components of the drug delivery device.

In an exemplary embodiment, the flexible arm may be arranged on one of the button or the clutch element and the toothed profile may be arranged on the other of the button and the clutch element. As described before, the button and the clutch element are both components of the drug delivery device, wherein the button is adapted to initiate a drug delivery mechanism.

Furthermore, the clutch element may be rotatable with respect to the button during dispensing the dose of medicament and during setting of the dose of medicament either the clutch element is rotationally locked to the button or the clutch element is only allowed a limited rotation relative to the button. Thus, the audible and/or tactile feedback is generated only during dose dispensing when relative movement between the clutch element and the button occurs. Rotationally locking the clutch element to the button during setting of the dose and unlocking it during dispensing prevents unintended manipulation of the set dose during dose dispensing.

In a further exemplary embodiment, the toothed profile comprises a ring with a number of ratchet teeth. The ring may be arranged on an inner circumference of the button, wherein the number of ratchet teeth projects radially inwards.

The flexible arm may be fixed to a proximal end of the clutch element, thereby extending axially in a distal direction. A deflecting axis of the flexible arm extends through the proximal end of the clutch element perpendicular to a longitudinal extension of the flexible arm in the distal direction.

Furthermore, the clutch element may comprise a clutch section configured as a hollow cylinder, wherein the flexible arm is arranged within a recess in a lateral surface of the clutch section. The recess is configured as a cut out in the lateral surface, wherein the flexible arm covers up to approximately 30 percent of the recess.

The flexible arm may further comprise a protrusion with at least one inclined surface. The protrusion is arranged on an end portion of the flexible arm, for example at the distal end, and is adapted to engage the ratchet teeth, in particular ratchet notches between the ratchet teeth. The protrusion may project radially outwards from an outer circumference of the clutch section.

In a further exemplary embodiment, the protrusion may include two angled faces, one of which may be inclined at a decreased (or shallower) angle than the other relative to the outer circumference of the clutch element. That means a torque needed to rotate the flexible arm relative to the toothed profile is smaller in a first rotating direction than in a second rotating direction. The first rotating direction is a direction in which the clutch element rotates relative to the button during dispensing the dose of medicament. During dose setting, the clutch element may rotate a limited amount relative to the button. It may rotate in the second rotating direction during setting of a dose, and in the first rotating direction if a user attempts to unset a selected dose. The different angles also mean that the click is louder in the first rotating direction.

Further scope of applicability of the present invention will become apparent from the detailed description given hereinafter. However, it should be understood that the detailed description and specific examples, while indicating exemplary embodiments of the invention, are given by way of illustration only, since various changes and modifications within the spirit and scope of the invention will become apparent to those skilled in the art from this detailed description.

### BRIEF DESCRIPTION OF THE FIGURES

The present invention will become more fully understood from the detailed description given herein below and the accompanying drawings which are given by way of illustration only, and thus, are not limitative of the present invention, and wherein:

FIG. 1 is a schematic view of an exemplary embodiment of a drug delivery device comprising a button, a dial grip and a ratchet mechanism,

FIG. 2 is a perspective view of an exemplary embodiment of a clutch element of a drug delivery device comprising a flexible arm, and

FIG. 3 is a perspective bottom view of an exemplary embodiment of a button of a drug delivery device comprising a toothed profile.

Corresponding parts are marked with the same reference symbols in all figures.

### DETAILED DESCRIPTION

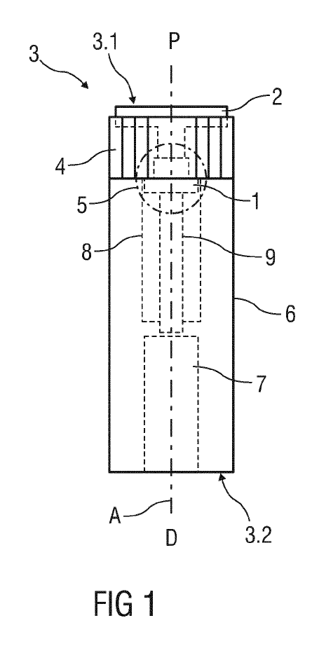

FIG. 1 shows a schematic view of a simplified embodiment of a drug delivery device 3 that may be configured as an injector pen. In particular, the drug delivery device 3 is configured as a pen-type device operable to deliver a variable, user-selectable dose of a medicament contained in the drug delivery device 3,

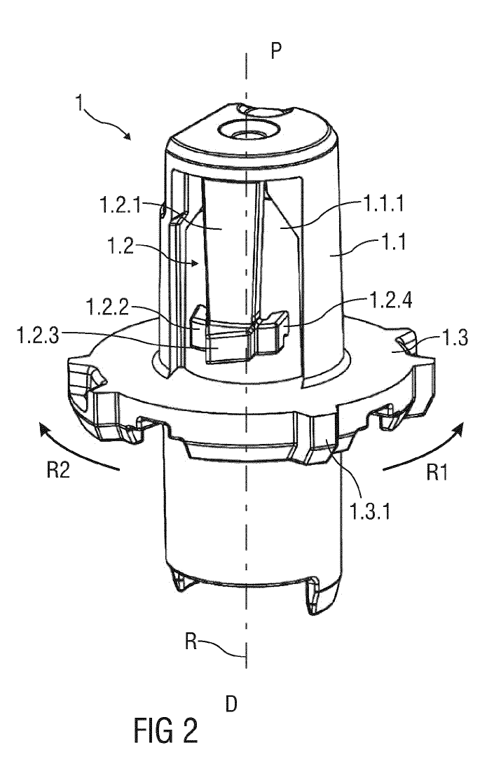

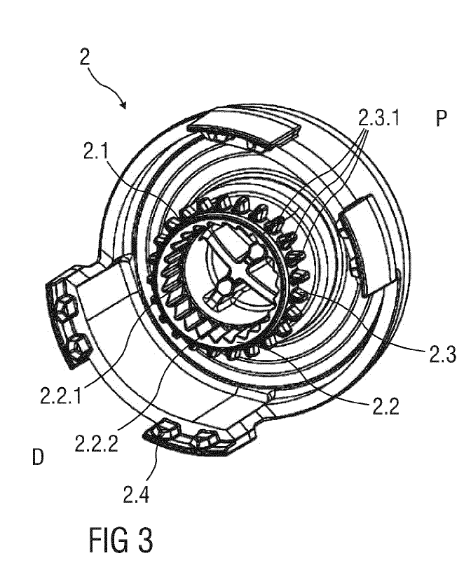

The drug delivery device 3 comprises a clutch element 1, a button 2, a dose selector component 4, a ratchet mechanism 5, which will be described in more detail in FIGS. 2 and 3, a housing 6, a cartridge 7 containing a medicament, a dose indicator sleeve 8 and a drive component 9.

The drug delivery device 3 further comprises a longitudinal axis A that extends axially from a proximal end 3.1 to a distal end 3.2. The proximal end 3.1 points towards a proximal direction P, whereby the proximal direction P refers to a direction that under use of the drug delivery device 3 is located the furthest away from a drug delivery site of a patient. Correspondingly, the distal end 3.2 points towards a distal direction D that refers to a direction that under use of the drug delivery device 3 is located closest to the drug delivery site of the patient.

The button 2 is arranged on the proximal end 3.1 of the drug delivery device 3 and is provided for initiating dose dispensing by depressing the button 2 in the distal direction D. The button 2 is permanently splined to the dose selector component 4 and provided with a central stem 2.1 (see FIG. 3) protruding in the distal direction D. The button 2 further comprises splines 2.3.1 (see FIG. 3) for engagement with the dose indicator sleeve 8 when the button 2 is not pressed. When the button 2 is pressed, the engagement will be released and the button 2 will be engaged to the housing 6 via further splines 2.4 (see FIG. 3). The latter engagement provides preventing rotation of the button 2 and hence the dose selector component 4 during dose dispensing. When the button 2 is not pressed, the engagement between the button 2 and the housing 6 is released, thus allowing a dose to be dialled.

The dose selector component 4 is a sleeve-like component with a serrated outer skirt as a gripping surface. The dose selector component 4 is axially constrained to the housing 6 and rotationally constrained to the button 2 as described above. For setting a dose of medicament, a user rotates the dose selector component 4 clockwise, which generates an identical rotation of the dose indicator sleeve 8.

The dose indicator sleeve 8 is a tubular, sleeve-like component and rotates during dose setting and dose correction (rotating counter-clockwise) due to the rotation of the dose selector component 4. Furthermore, the dose indicator sleeve 8 rotates during dose dispensing, for example due to the load of a spring (not shown), which is loaded during dose setting. The dose indicator sleeve 8 may be provided with a sequence of numbers each representing a dose of medicament. The selected dose may be visible through an aperture arranged within the housing 6.

The drive component 9 is a tubular, sleeve-like component and arranged within the dose indicator sleeve 8. Within the drive component 9, a piston rod (not shown) may be arranged that is rotationally constrained to the drive component 9. The drive component 9 is axially movable with respect to the housing 6, the dose indicator sleeve 8 and the piston rod. In particular, the drive component 9 is prevented for rotation relative to the housing 6 during dose setting, for example via further spline features (not shown). When the button 2 is pressed, the drive component 9 disengages from the housing 6 in a manner rotating relative to the housing 6. During dose dispensing, the drive component 9 is engaged to the dose indicator sleeve 8 in manner that the drive component 9 rotates together with the dose indicator sleeve 8.

The clutch element 1 is a connective link between the dose indicator sleeve 8, the drive component 9 and the button 2. The clutch element 1 is engaged to the dose indicator sleeve 8 via projections 1.3.1 (see FIG. 2) and to the drive component 9 for example via a ratchet interface (not shown). The ratchet interface provides a detented position between the dose indicator sleeve 8 and drive component 9 corresponding to each dose unit.

In order to perform dose dispensing, the drug delivery device 3 may be operated according to the following exemplary method.

The user selects a variable dose of liquid medicament by rotating the dose selector component 4 clockwise, which generates an identical rotation in the dose indicator sleeve 8 relative to the drive component 9. The rotation of the dose indicator sleeve 8 may result in charging of a spring (not shown), increasing the energy stored within it. Furthermore, as the dose indicator sleeve 8 rotates, a gauge element (not shown) may be translated in a manner showing the value of the dialled dose. The user may then choose to increase the selected dose by continuing to rotate the dose selector component 4 in the clockwise direction or to correct the dose by rotating the dose selector component 4 in the counter-clockwise direction.

When a dose has been selected, the user is able to activate dose by depressing the button 2 axially in the distal direction D.

When the button 2 is depressed, the button 2 disengages from the dose indicator sleeve 8, thus the button 2 and the dose selector component 4 are rotationally disengaged from the dose dispense mechanism. The button 2 engages with the housing 6 via the further splines 2.4 (see FIG. 3), thereby preventing rotation of the button 2 and hence the dose selector component 4 during dose dispensing.

By depressing the button 2, the clutch element 1 and the drive component 9 travel axially in the distal direction D together with the button 2. This causes an engagement between the drive component 9 and the dose indicator sleeve 8, thereby, preventing relative rotation between the drive component 9 and the dose indicator sleeve 8 during dose dispensing. Furthermore, the drive component 9 disengages from the housing 6 and the drive component 9 is allowed for rotating relative to the housing 6. The drive component 9 may be driven by the spring (mentioned before) that drives the dose indicator sleeve 8 and the clutch element 1 as well.

The rotational movement of the drive component 8 relative to the housing 6 causes the piston rod to rotate with relative to the housing 6. For example, the piston rod is splined to the drive component 8 and threaded to the housing 6. For example, the piston rod is threaded to a part of the housing 6 protruding through a distal end of the drive component 8. Due to the threaded engagement to the housing 6, the piston rod will be moved axially in the distal direction D with respect to the housing 6 into the cartridge 7. The piston rod may be coupled to a bung on a distal end that is provided to expel the medicament out of the cartridge 7. A distal end of the cartridge 7 may be provided with a needle (not shown) through which the medicament may be expelled out of the cartridge 7.

Once the delivery of a dose is stopped, the user may release the button 2, which will re-engage the drive component 9 and the housing 6. The dose dispense mechanism is now returned to an ‘at rest’ condition.

To provide audible feedback during dose dispense, the drug delivery comprises the ratchet mechanism 5 that is coupled to the button 2 and the clutch element 1. These components will be described in more detail below.

FIG. 2 shows a perspective view of an exemplary embodiment of a clutch element 1 of the drug delivery device 3.

The clutch element 1 comprises a substantially hollow cylindrical clutch section 1.1 including a flexible arm 1.2 and a clutch plate 1.3.

The clutch section 1.1 extends axially between the proximal direction P and the distal direction D. A part of the clutch section 1.1 that is located proximally behind the clutch plate 1.3 comprises a recess 1.1.1 that is partly covered by the flexible arm 1.2. According to the present embodiment, the recess 1.1.1 is configured as a cut-out of a lateral surface of the clutch section 1.1 that covers approximately one-third of the lateral surface of the proximal part of the clutch section 1.1. Alternatively, the recess 1.1.1 may be configured with less or more dimensions than shown in figure ‘2.

The flexible arm 1.2 is fixed on a proximal end of the clutch section 1.1 and extends in the distal direction D facing the clutch plate 1.3 in a predetermined distance. The flexible arm 1.2 comprises a proximal arm section 1.2.1 and a distal arm section 1.2.2, wherein the proximal arm section 1.2.1 is an elongate part of the flexible arm 1.2 extending in the distal direction D. The distal arm section 1.2.2 is a free end of the flexible arm 1.2 and extends perpendicular to the proximal arm section 1.2.1.

The flexible arm 1.2 further comprises a protrusion 1.2.3 with an inclined surface that projects out of the recess 1.1.1. In particular, the protrusion 1.2.3 includes two angled faces, one of which is inclined at a decreased (or shallower) angle than the other relative to the outer circumference of clutch element 1.

The clutch element 1 rotates relative to the button 2 during dose setting and dose dispensing.

The clutch element 1 thus comprises a rotational axis R that runs parallel to a longitudinal extension of the flexible arm 1.2. The rotational axis R defines two rotational directions R1, R2.

A first rotating direction R1 is a direction in which the clutch element 1 rotates relative to the button 2 during drug delivery, i. e. counter-clockwise. During dose setting, the clutch element 1 may rotate a limited amount relative to the button 2. It may rotate in the second rotating direction R2 during setting of a dose, i. e. clockwise, and in the first rotating direction R1 if a user attempts to unset or correct a selected dose which will be described in more detail in FIG. 3. The function of the clutch element 1 during dose dispense will be described in more detail in FIG. 3 as well.

Furthermore, the clutch element 1 comprises a number of projections 1.3.1 that are arranged around an outer circumference of the clutch plate 1.3. The projections 1.3.1 are adapted to engage with the dose indicator sleeve 8 in order to prevent rotation of the clutch element 1 with respect to the dose indicator sleeve 8.

FIG. 3 shows an exemplary embodiment of a bottom view of the button 2 of the drug delivery device 3 that is mechanically engaged with the clutch element 1 as described in FIG. 2.

The button 2 forms a proximal end of the drug delivery device 3, wherein FIG. 3 shows a distal side of the button 2 that is directed towards the clutch element 1.

The button 2 comprises the central stem 2.1 protruding in the distal direction D. The stem 2.1 is hollow and includes a toothed profile 2.2 on an inner circumference configured as a ring of ratchet teeth 2.2.1 and ratchet notches 2.2.2, wherein one ratchet notch 2.2.2 is arranged between two ratchet teeth 2.2.1 respectively. The ratchet teeth 2.2.1 and thus the ratchet notches 2.2.2 respectively comprise an asymmetric form that differs from an equilateral triangle. In particular, each tooth 2.2.1 has a moderate slope on one edge and a much steeper slope on the other edge. As the button 2 is stationary during dose dispense, this can be used for providing audible and/or tactile feedback during dose dispensing. For this, the ratchet mechanism 5 is provided, which comprises the flexible arm 1.2 and the toothed profile 2.2. In particular, the protrusion 1.2.3 positively engages the ratchet notches 2.2.2 during dose dispense in order to generate an audible and/or tactile feedback for the user as described further below.

The stem 2.1 further includes a spline profile 2.3 on an outer circumference configured as the ring of splines 2.3.1 (mentioned in FIG. 1) that may engage clutch features (not shown) of the dose indicator sleeve 8 during dose setting and dose correction. The button 2 further comprises a discontinuous annular skirt with the further splines 2.4 for engaging further clutch features on the housing 6in order to prevent rotation of the button 2 with respect to the housing 6 during dose dispense.

By engagement of the clutch element 1 and the button 2, the stem 2.1 is arranged over the proximal part of the clutch section 1.1, wherein a distal end of the stem 2.1 engages a proximal side of the clutch plate 1.3. Hence, the toothed profile 2.2 in the inner circumference of the stem 2.1 faces the lateral surface of the clutch section 1.1 in the level of the protrusion 1.2.3.

As mentioned before, the ratchet mechanism 5 comprises the flexible arm 1.2 and the toothed profile 2.2 and is provided to generate the audible and/or tactile feedback. The ratchet mechanism 5 will be described in more detail below in context with the description of an exemplary dose dispense process.

At the beginning of the drug delivery process, a user may prime the drug delivery device 3 by selecting a certain dose of medicament. This is performed by rotating the dose selector component 4 clockwise. During dose setting, the button 2 and the clutch element 1 are nominally locked against rotation relative to each other, although a limited amount of relative rotation may be possible.

There are clearances included between different parts in the described mechanism, in order to improve the tactile quality of the selecting or dialling experience of the user. For example, there are rotational clearances between the dose selector component 4 and the button 2, the button 2 and the dose indicator sleeve 8, and the dose indicator sleeve 8 and the projections 1.3.1 on the clutch element 1. This allows the button 2 to rotate slightly relative to the clutch element 1 when the user rotates the dose setting component 4 during dose setting.

As a result, the protrusion 1.2.3 of the flexible arm 1.2 may engage another ratchet notch 2.2.2. If the user then attempts to change the selected dose, some torque may be transmitted from the toothed profile 2.2 to the protrusion 1.2.3. However, the clutch element 1 can be rotated a limited amount with respect to the button 2 both in the first rotation direction R1 and in the second rotation direction R2 without being mechanically damaged, because the design of the flexible arm 1.2 allows it to flex inwards at a relatively small torque, thus limiting the force applied to the flexible arm 1.2.3. This is particularly due to the length of the lever arm from the root of the flexible arm 1.2 to its tip where protrusion 1.2.3 contacts the toothed profile 2.2, its axial direction, and the angle of the contact surface that provides a radial component of force to cause the flexible arm 2.1 to deflect inwards. Hence, an unset or correction of a selected dose may be performed without losing the audible and/or tactile feedback during dose dispense.

If a dose has been selected, the user is able to activate a delivery mechanism to start drug delivery. Dispensing the dose is initiated by the user by depressing the button 2 axially in the distal direction D, wherein the clutch element 1 is moved axially as well. When the button 2 is depressed, the splines 2.3.1 of the button 2 and the clutch features of the dose indicator sleeve 8 are disengaged, thereby rotationally disconnecting the button 2 and the dose selector component 4 from the dose dispensing mechanism. The further splines 2.4 on the button 2 engage with the further clutch features on the housing 6, thereby preventing rotation of the button 2 relative to the housing 6 during dose dispense as it is already described in FIG. 1.

As the button 2 is locked against rotation with respect to the housing 6 and the clutch element 1 is engaged with the dose indicator sleeve 8 and the dose indicator sleeve 8 rotates in the first rotational direction R1 during dose dispense, the clutch element 1 rotates in the first rotational direction R1 with respect to the button 2. The protrusion 1.2.3 engages the toothed profile 2.2 to produce an audible and/or tactile click with each dose increment delivered. When the clutch element 1 rotates relative to the button 2, contact between the protrusion 1.2.3 and the ratchet tooth 2.2.1 forces the flexible arm 1.2 to deflect radially inwards until the tip of the protrusion 1.2.3 contacts the tip of the ratchet tooth 2.2.1. Further relative rotation allows the flexible arm 1.2 to relax radially outwards.

Audible feedback is provided when the protrusion 1.2.3 contacts the ratchet notch 2.2.2. The torque that needs to be applied to clutch element 1 before the flexible arm 1.2 deflects depending on a ratio of a thickness and width of the flexible arm 1.2, an angle of the inclined surface of the protrusion 1.2.3 and a form of the ratchet teeth 2.2.1. The angle of the inclined surfaces that contact during rotation of the clutch element 1 in the first rotating direction R1 make a shallow angle relative to the outer circumference of clutch element 1.2.1, so beneficially reducing the torque needed during dispense.

Once the delivery of a dose is stopped and the dose indicator sleeve 8 indicates a zero dose, the user may release the button 2. The drug delivery device 3 may be provided with an audible and/or tactile feature at the end of drug delivery.

The term “drug” or “medicament”, as used herein, means a pharmaceutical formulation containing at least one pharmaceutically active compound, wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser- Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

Those of skill in the art will understand that modifications (additions and/or removals) of various components of the apparatuses, methods and/or systems and embodiments described herein may be made without departing from the full scope and spirit of the present invention, which encompass such modifications and any and all equivalents thereof.

### LIST OF REFERENCES

1 clutch element

1.1 clutch section

1.1.1 recess

1.2 flexible arm

1.2.1 proximal arm section

1.2.2 distal arm section

1.2.3 protrusion

1.3 clutch plate

1.3.1 projection

2 button

2.1 stem

2.2 toothed profile

2.2.1 ratchet tooth

2.2.2 ratchet notch

2.3 spline profile

2.3.1 splines

2.4 further splines

3 drug delivery device

3.1 proximal end

3.2 distal end

4 dial grip

5 ratchet mechanism

6 housing

7 cartridge

8 dose indicator sleeve

9 drive component

A longitudinal axis

D distal direction

P proximal direction

R rotational axis

R1 first rotating direction

R2 second rotating direction

1-14. (canceled)

15. A drug delivery device comprising:
a dose selector component operable to set a dose of a medicament,
a depressible button operable to initiate dispensing the dose of the medicament,
a drive component adapted to dispense the medicament, and
a clutch element arranged between the dose selector component and the drive component.
a ratchet mechanism comprising a flexible arm and a toothed profile, an end portion of the flexible arm being configured to engage with the toothed profile and to perform a relative movement thereto while generating an audible and/or tactile feedback, wherein the flexible arm extends in a plane perpendicular to a direction of relative movement between the flexible arm and the toothed profile.

16. The drug delivery device according to claim 15, wherein:
a free end of the flexible arm is configured to engage with the toothed profile and to perform a rotational movement relative to the toothed profile during the dispensing, thereby generating the audible and/or tactile feedback, and
the flexible arm extends in a plane parallel to a rotational axis of the flexible arm.

17. The drug delivery device according to claim 15, further comprising a longitudinal axis extending from a proximal end towards a distal end, wherein a rotational axis of the flexible arm is aligned with the longitudinal axis.

18. The drug delivery device according to claim 15, wherein the flexible arm is arranged on one of the button or the clutch element and the toothed profile is arranged on the other of the button and the clutch element.

19. The drug delivery device according to claim 15, wherein:
the clutch element is rotatable with respect to the button during the dispensing, and
the clutch element and the button are configured such that relative rotation between the clutch element and the button is limited during setting the dose of the medicament.

20. The drug delivery device according to claim 15, further comprising:
a housing, and
a cartridge adapted to contain the medicament and received within the housing,
wherein the drive component is adapted to push the medicament out of the cartridge.

21. A drug delivery device for mechanically dispensing a dose of a medicament, the drug delivery device comprising:
a ratchet mechanism adapted to generate an audible and/or tactile feedback during the dispensing,
wherein the ratchet mechanism comprises a flexible arm and a toothed profile, an end portion of the flexible arm being configured to engage with the toothed profile and to perform a relative movement thereto while generating the audible and/or tactile feedback, and
wherein the flexible arm extends in a plane perpendicular to a direction of the relative movement between the flexible arm and the toothed profile.

22. The drug delivery device according to claim 21, wherein:
a free end of the flexible arm is configured to engage with the toothed profile and to perform a rotational movement relative to the toothed profile during the dispensing, thereby generating the audible and/or tactile feedback, and
the flexible arm extends in a plane parallel to a rotational axis of the flexible arm.

23. The drug delivery device according to claim 21, further comprising a longitudinal axis extending from a proximal end towards a distal end, wherein a rotational axis of the flexible arm is aligned with the longitudinal axis.

24. The drug delivery device according to claim 21, further comprising:
a housing,
a cartridge adapted to contain the medicament and received within the housing,
a depressible button operable to initiate the dispensing,
a dose selector component operable to set the dose of the medicament or unset another dose of medicament before the dispensing,
a drive component adapted to push the medicament out of the cartridge, and
a clutch element arranged between the dose selector component and the drive component.

25. The drug delivery device according to claim 24, wherein the flexible arm is arranged on one of the button or the clutch element and the toothed profile is arranged on the other of the button and the clutch element.

26. The drug delivery device according to claim 24, wherein:
the clutch element is rotatable with respect to the button during the dispensing, and
the clutch element and the button are configured such that relative rotation between the clutch element and the button is limited during setting the dose of medicament.

27. The drug delivery device according to claim 21, wherein the toothed profile comprises a ring with a number of ratchet teeth.

28. The drug delivery device according to claim 27, further comprising a button to initiate the dispensing, wherein a number of ratchet teeth is arranged on an inner circumference of the button, thereby projecting radially inwards.

29. The drug delivery device according to claim 27, wherein the flexible arm comprises a protrusion with at least one inclined surface that is arranged on a distal arm section and adapted to engage the ratchet teeth.

30. The drug delivery device according to claim 29, further comprising a clutch element arranged between a dose selector component and a drive component, wherein the protrusion projects radially outwards from an outer circumference of a clutch section of the clutch element.

31. The drug delivery device according to claim 29, further comprising a clutch element arranged between a dose selector component and a drive component,
wherein the protrusion comprises two inclined surfaces, a first of the two inclined surfaces comprising an angle relative to an outer circumference of the clutch element less than an angle between a second of the two inclined surfaces and the outer circumference of the clutch element.

32. The drug delivery device according to claim 15, further comprising a clutch element arranged between a dose selector component and a drive component,
wherein the flexible arm is fixed to a proximal end of the clutch element, thereby extending in a distal direction.

33. The drug delivery device according to claim 21, further comprising a clutch element arranged between a dose selector component and a drive component,
wherein the clutch element comprises a clutch section comprising a hollow cylinder, wherein the flexible arm is arranged within a recess in a lateral surface of the clutch section.

34. The drug delivery device according to claim 21, wherein the ratchet mechanism is configured such that a torque needed to deflect the flexible arm is smaller in a first rotating direction than in a second rotating direction.
